(19) United States
(12) Patent Application Publication
(10) Pub. No.: US 2023/0405308 A1
(43) Pub. Date: Dec. 21, 2023

(54) IMPLANTABLE MEDICAL SYSTEMS AND LEADS WITH FRINGE ELECTRODES FOR SENSING BIOMARKER SIGNALS

(71) Applicant: MEDTRONIC, INC., Minneapolis, MN (US)

(72) Inventor: Janardan Vaidyanathan, Thane (IN)

(21) Appl. No.: 18/207,599

(22) Filed: Jun. 8, 2023

Related U.S. Application Data
(60) Provisional application No. 63/353,023, filed on Jun. 16, 2022.

(51) Int. Cl. A61N 1/05; A61N 1/36; A61B 90/00

(52) U.S. Cl. CPC A61N 1/0534; A61N 1/36139; A61B 90/39; A61B 2090/3966

(57) ABSTRACT

Fringe sensing electrodes are provided on implantable medical leads so that a fringe sensing electrode is present in a more proximal position and/or more distal position than all stimulation electrodes. By having a fringe sensing electrode more proximal than all stimulation electrodes and another fringe sensing electrode more distal than all stimulation electrodes, all stimulation electrodes are available for providing stimulation signals while still being surrounded by sensing electrodes capable for sensing resulting biomarker signals. Additionally, the fringe sensing electrodes may serve as radiopaque markers to determine implanted lead position and/or radiopaque orientation markers to determine segmented electrode orientation of an implanted lead.

### RELATED APPLICATIONS

The present application claims priority to U.S. Provisional Application No. 63/353,023, filed on Jun. 16, 2022, and titled IMPLANTABLE MEDICAL SYSTEMS AND LEADS WITH FRINGE ELECTRODES FOR SENSING BIOMARKER SIGNALS.

### TECHNICAL FIELD

Embodiments relate to implantable medical systems and leads for providing therapy to patients. More particularly, embodiments relate to implantable medical systems and leads that include fringe electrodes for sensing and detection of biomarkers.

### BACKGROUND

Implantable medical systems which may include components that may be external or implantable provide a particular therapy for a patient suffering from a condition that electrical stimulation may address. For instance, certain neurological conditions such as chronic pain, epilepsy, headache, psychiatric disorders, memory dysfunction, urinary or fecal incontinence, sexual dysfunction, obesity and eating disorders, gastroparesis and movement disorders including Parkinson's disease, essential tremor, dystonia, and the like can be addressed by deep brain stimulation therapy. This therapy is provided by an implantable medical system that includes an external or implantable medical device that produces electrical stimulation and an implantable medical lead that delivers the electrical stimulation to a target location within the neural tissue of the patient. In certain examples for delivering stimulation via one or more leads that includes electrodes located proximate to target locations associated with the brain, spinal cord, pelvic nerves, peripheral nerves, or the gastrointestinal track. Electrical stimulation can be used in a variety of therapeutic applications such as deep brain stimulation (DBS), occipital nerve stimulation (ONS), motor cortex stimulation (MCS), spinal cord stimulation (SCS), pelvic stimulation, sacral nerve stimulation (SNS), phrenic nerve stimulation, gastric stimulation, or peripheral nerve field stimulation (PNFS).

When implanting the medical lead to the target site within the body of the patient, such as within the brain, the precise site at the general target location where stimulation therapy could be delivered to get the optimal result may be difficult to know in advance of initiating the therapy. One manner of dealing with this difficulty is to include several electrodes on the lead separated over the length of the lead. Then, different electrodes along the length may be utilized in providing the stimulation therapy to find the optimal one to use.

However, a limitation can arise where sensing of electrical biomarker signals is desired to gather information that helps in determining the efficacy of stimulation from any particular electrode. It is preferable to provide sensing electrodes on both sides of the stimulation electrode(s) in order to best capture the biomarker signals that provide the efficacy information desired. This aids in providing simultaneous sensing and stimulation possibilities with a stimulus artifact blanking approach for example. For a particular lead implantation, the optimal electrode for stimulation may be an electrode located at one extreme of the electrode set, such as the most distal or most proximal electrode. In that case, there is no additional electrode available to surround the stimulation electrode since the stimulation electrode occupies the most extreme position. Thus, sensing with electrodes that surround the stimulation electrode is not possible and the detection of the biomarker signal is thereby hindered. For example, in some instances biomarker source localization or determination may not be possible as the sensed information is of limited value.

### SUMMARY

Embodiments address issues such as these and others by providing at least one passive fringe electrode at a most extreme position at the distal end of the lead not already occupied by a stimulation capable electrode, namely the most proximal position and/or the most distal position at the distal end of the lead. The one or more passive fringe electrodes may be directly coupled to the sensing module of the implantable medical device and serve no stimulation function. Furthermore, one or more of the passive fringe electrodes may serve as a radiopaque marker and may further be fragmented and aligned with stimulation electrode segments to further serve as a radiopaque orientation marker for identifying the position of the stimulation electrode segments.

Embodiments provide an implantable medical lead configured for implantation within a patient. The implantable medical lead includes a lead body, a first plurality of proximal contacts on a proximal end of the lead body, and a second plurality of proximal contacts on the proximal end of the lead body. The implantable medical lead further includes at least one distal electrode on a distal end of the lead body, the at least one distal electrode comprising a segmented electrode having segments separated circumferentially about the lead body with each of the segments having at least one edge at circumferential positions about the lead body. A first sensing electrode is located on the distal end of the lead body, the first sensing electrode being radiopaque and being located at a first longitudinal position of the distal end of the lead body relative to the at least one distal electrode. The first sensing electrode includes electrode fragments having characteristics that are visually distinguishable from each other and where the characteristics have an orientation feature that aligns circumferentially with an aspect of a corresponding one of the segments of the segmented electrode. A second sensing electrode is located at a second longitudinal position of the distal end of the lead body so that the at least one distal electrode is longitudinally between the first sensing electrode and the second sensing electrode. A first plurality of conductors is surrounded by the lead body and extend longitudinally, each conductor of the first plurality of conductors interconnecting one of the proximal contacts of the first plurality of proximal contacts to one of the segments of the segmented electrode on the distal end of the lead body. A second plurality of conductors are surrounded by the lead body and extend longitudinally. A first conductor of the second plurality of conductors interconnects one of the proximal connectors of the second plurality of proximal connectors to the first sensing electrode and a second conductor of the second plurality of conductors interconnects another one of the proximal connectors of the second plurality of proximal connectors to the second sensing electrode.

Embodiments provide an implantable medical system configured to provide stimulation and sensing within a patient. The implantable medical system includes an implantable medical device that includes a stimulation module, a sensing module, a first plurality of contacts electrically coupled to the stimulation module, and a second plurality of contacts coupled to the sensing module. The implantable medical system further includes an implantable medical lead that includes a lead body and a first plurality of proximal connectors on a proximal end of the lead body, each proximal connector of the first plurality of proximal connectors being coupled to a corresponding proximal contact that is coupled to the stimulation module. The implantable medical lead includes a second plurality of proximal connectors on the proximal end of the lead body, each proximal connector of the second plurality of proximal connectors being coupled to a corresponding proximal contact that is coupled to the sensing module. At least one distal electrode is located on a distal end of the lead body, and the at least one distal electrode includes a segmented electrode having segments separated circumferentially about the lead body with each of the segments having at least one edge at circumferential positions about the lead body. A first sensing electrode is located on the distal end of the lead body, and the first sensing electrode is radiopaque and is located at a first longitudinal position of the distal end of the lead body relative to the at least one distal electrode. The first sensing electrode includes electrode fragments having characteristics that are visually distinguishable from each other and where the characteristics have an orientation feature that aligns circumferentially with an aspect of a corresponding one of the segments of the segmented electrode. A second sensing electrode is located at a second longitudinal position of the distal end of the lead body so that the at least one distal electrode is longitudinally between the first sensing electrode and the second sensing electrode. A first plurality of conductors is surrounded by the lead body and extend longitudinally, each conductor of the first plurality of conductors interconnecting one of the proximal contacts of the first plurality of proximal contacts to one of the segments of the segmented electrode on the distal end of the lead body. A second plurality of conductors are surrounded by the lead body and extend longitudinally. A first conductor of the second plurality of conductors interconnects one of the proximal connectors of the second plurality of proximal connectors to the first sensing electrode and a second conductor of the second plurality of conductors interconnects another one of the proximal connectors of the second plurality of proximal connectors to the second sensing electrode.

Embodiments provide an implantable medical system configured to provide stimulation and sensing within a patient that includes an implantable medical device that includes a stimulation module, a sensing module, a switching module coupled to the stimulation module and the sensing module, a plurality of contacts signal coupled to the switching module, and a plurality of contacts coupled to the sensing module. The implantable medical system further includes an implantable medical lead that includes a lead body and a first plurality of proximal connectors on a proximal end of the lead body. Each proximal connector of the first plurality of proximal connectors is coupled to a corresponding proximal contact that is coupled to the switching module. A second plurality of proximal connectors is located on the proximal end of the lead body, each proximal connector of the second plurality of proximal connectors being coupled to a corresponding proximal contact that is directly coupled to the sensing module. At least one distal electrode on a distal end of the lead body, the at least one distal electrode includes a segmented electrode having segments separated circumferentially about the lead body with each of the segments having at least one edge at circumferential positions about the lead body. A first sensing electrode is located on the distal end of the lead body, and the first sensing electrode is located at a first longitudinal position of the distal end of the lead body relative to the at least one distal electrode. A second sensing electrode is located at a second longitudinal position of the distal end of the lead body so that the at least one distal electrode is longitudinally between the first sensing electrode and the second sensing electrode. A first plurality of conductors is surrounded by the lead body and extend longitudinally, and each conductor of the first plurality of conductors interconnects one of the proximal contacts of the first plurality of proximal contacts to one of the segments of the segmented electrode on the distal end of the lead body. A second plurality of conductors is surrounded by the lead body and extending longitudinally, and a first conductor of the second plurality of conductors interconnects one of the proximal connectors of the second plurality of proximal connectors to the first sensing electrode. A second conductor of the second plurality of conductors interconnects another one of the proximal connectors of the second plurality of proximal connectors to the second sensing electrode.

Embodiments provide a method of providing stimulation and sensing within a patient. The method involves providing an implantable medical device that includes a stimulation module, a sensing module, a plurality of contacts signal coupled to the stimulation module, and a plurality of contacts coupled to the sensing module. The method involves providing an implantable medical lead that includes a lead body and a first plurality of proximal connectors on a proximal end of the lead body, where each proximal connector of the first plurality of proximal connectors is coupled to a corresponding proximal contact that is coupled to the stimulation module. The implantable medical lead being provided further includes a second plurality of proximal connectors on the proximal end of the lead body, each proximal connector of the second plurality of proximal connectors being coupled to a corresponding proximal contact that is coupled to the sensing module. The implantable medical lead being provided further includes at least one distal electrode on a distal end of the lead body, the at least one distal electrode comprising a segmented electrode having segments separated circumferentially about the lead body with each of the segments having at least one edge at circumferential positions about the lead body. The implantable medical lead being provided includes a first sensing electrode on the distal end of the lead body, and the sensing electrode is radiopaque and is located at a first longitudinal position of the distal end of the lead body relative to the at least one distal electrode. The first sensing electrode includes electrode fragments having characteristics such as shape, orientation, or position that are visually distinguishable from each other and where the characteristics have an orientation feature that aligns circumferentially with an aspect of a corresponding one of the segments of the segmented electrode. The implantable medical device being provided includes a second sensing electrode located at a second longitudinal position of the distal end of the lead body so that the at least one distal electrode is longitudinally between the first sensing electrode and the second sensing electrode. The implantable medical device being provided includes a first plurality of conductors surrounded by the lead body and extending longitudinally, each conductor of the first plurality of conductors interconnecting one of the proximal contacts of the first plurality of proximal contacts to one of the segments of the segmented electrode on the distal end of the lead body. The implantable medical device being provided includes a second plurality of conductors surrounded by the lead body and extending longitudinally, where a first conductor of the second plurality of conductors interconnects one of the proximal connectors of the second plurality of proximal connectors to the first sensing electrode and a second conductor of the second plurality of conductors interconnects another one of the proximal connectors of the second plurality of proximal connectors to the second sensing electrode. The method further involves determining which segment of the segmented electrode to provide a stimulation signal based on the location of the orientation feature of each of the electrode segments of the first sensing electrode. Additionally, the method involves providing the stimulation signal from the stimulation module to the determined segment of the segmented electrode; and sensing at the sensing module for a biomarker signal occurring between the first sensing electrode and the second sensing electrode.

Embodiments provide a method of providing stimulation and sensing within a patient that involves providing an implantable medical device comprising a stimulation module, a sensing module, a switching module coupled to the stimulation module and the sensing module, a plurality of contacts signal coupled to the switching module, and a plurality of contacts coupled to the sensing module. The method further involves providing an implantable medical lead that includes a lead body and a first plurality of proximal connectors on a proximal end of the lead body, where each proximal connector of the first plurality of proximal connectors is coupled to a corresponding proximal contact that is coupled to the switching module. The implantable medical lead being provided further includes a second plurality of proximal connectors on the proximal end of the lead body, where each proximal connector of the second plurality of proximal connectors is coupled to a corresponding proximal contact that is directly coupled to the sensing module. The implantable medical lead being provided further includes at least one distal electrode on a distal end of the lead body, and the at least one distal electrode includes a segmented electrode having segments separated circumferentially about the lead body with each of the segments having at least one edge at circumferential positions about the lead body. The implantable medical lead being provided further includes a first sensing electrode on the distal end of the lead body, where the first sensing electrode is located at a first longitudinal position of the distal end of the lead body relative to the at least one distal electrode. The implantable medical lead being provided further includes a second sensing electrode located at a second longitudinal position of the distal end of the lead body so that the at least one distal electrode is longitudinally between the first sensing electrode and the second sensing electrode. The implantable medical lead being provided further includes a first plurality of conductors surrounded by the lead body and extending longitudinally, where each conductor of the first plurality of conductors interconnects one of the proximal contacts of the first plurality of proximal contacts to one of the segments of the segmented electrode on the distal end of the lead body. The implantable medical lead being provided further includes a second plurality of conductors surrounded by the lead body and extending longitudinally, where a first conductor of the second plurality of conductors interconnects one of the proximal connectors of the second plurality of proximal connectors to the first sensing electrode and a second conductor of the second plurality of conductors interconnects another one of the proximal connectors of the second plurality of proximal connectors to the second sensing electrode. The method further involves configuring the switching module to signal connect a contact of the implantable medical device that corresponds to a desired segment of the segmented electrode to the stimulation module. The method further involves providing a stimulation signal from the stimulation module to the desired segment of the segmented electrode, and sensing at the sensing module for a biomarker signal occurring between the first sensing electrode and the second sensing electrode.

### DESCRIPTION OF THE DRAWINGS

FIG. 1 shows an example of an implantable medical system including an implantable medical lead with fringe sensing electrodes.

FIG. 2 shows a first example of a distal end of the implantable medical lead with fringe sensing electrodes.

FIG. 3A shows a second example of a distal end of the implantable medical lead with fringe sensing electrodes.

FIG. 3B shows a third example of a distal end of the implantable medical lead with fringe sensing electrodes.

FIG. 4 shows a fourth example of a distal end of the implantable medical lead with fringe sensing electrodes.

FIG. 5 shows a fifth example of a distal end of the implantable medical lead with fringe sensing electrodes.

FIG. 6 shows an example of operations performed by an implantable medical device of the implantable medical system in conjunction with the implantable medical lead that has fringe sensing electrodes.

### DETAILED DESCRIPTION

Embodiments include implantable medical leads and implantable medical systems that include implantable medical leads where the implantable medical leads include fringe sensing electrodes. Furthermore, embodiments provide radiopaque markers configured as fringe sensing electrodes as well as radiopaque orientation markers configured as fringe sensing electrodes to allow the fringe sensing electrodes to provide multiple purposes.

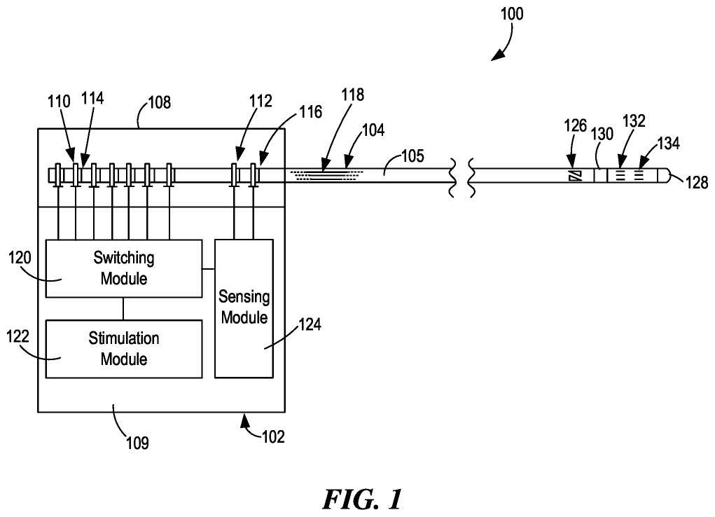

FIG. 1 shows an example of an implantable medical system 100 that provides stimulation and sensing functions when delivering therapy to a patient. The implantable medical system 100 includes an implantable medical device 102 and an implantable medical lead 104 having an insulative lead body 105 with a proximal end coupled to the implantable medical device 102 while having electrodes including fringe sensing electrodes on a distal end. The coupling of the lead 104 to the device 102 may be a direct coupling where the lead 104 is directly inserted into a lead receptacle of the device 102 or may be indirect where the lead 104 directly couples to a lead extension and the lead extension directly couples to the device 102. The implantable medical system 100 may of any type that provides a necessary therapy to the patient. It is particularly effective for an implantable medical system 100 that provides deep brain stimulation therapy to include the fringe sensing electrodes on the lead that is positioned within the brain of the patient.

The implantable medical device of this example includes both a stimulation module 122 for producing stimulation signals and a sensing module 124 for sensing biomarker signals within the patient in proximity of a target location including those evoked (e.g., such as a resonant response) or modulated by the introduction of the stimulation signals at the target location within the patient. A switching module 120 may be present to connect conduction paths to the distal end of the lead 104 to either the stimulation module 122 or the sensing module 124. This allows selection of the distal electrodes that produce the optimal therapy due to their location for delivering the stimulation signal. This also allows selection of the distal electrodes that sense the relevant biomarker signal. However, the sensing module 124 is also directly coupled to conduction paths to the fringe sensing electrodes to selectively establish a particular fringe sensing electrode as a passive sensing electrode. The appropriate fringe sensing electrode can be selected based on which non-fringe electrode(s) is/are selected for stimulation and together with an electrode outflanking the stimulation electrode(s) on a side opposite from the fringe sensing electrode provides a differential bipolar sensing montage. The fringe sensing electrode may be established as a reference electrode while a non-fringe sensing electrode is established as an active sensing electrode. It will be appreciated that the switching module 120 may be coupled to the stimulation module and sensing module via electrical coupling or alternatively an optical coupling to provide further isolation.

Non-fringe electrodes present on the lead 104 can be used either for sensing or stimulation. While using biomarker guided programming when setting parameters such as amplitude, pulse width and frequency or selecting a configuration such as monopolar or bipolar, it may be necessary to determine the source of a biomarker signal to modulate a feature of the same (e.g., local field potentials suppressing the beta band and evoking the gamma band in a Parkinson's disease patient). The non-fringe electrode being outflanked by another sensing electrode allows the user to determine the source of the biomarker signal.

These distal electrodes are also shown in FIG. 1 for the lead 104. A first fringe sensing electrode 126 that is ultimately directly coupled to the sensing module 124 resides in a most proximal location on the distal end of the lead 104. The first fringe sensing electrode 126 may be constructed of a radiopaque biocompatible conductive material, such as a platinum-iridium alloy, to serve as a radiopaque marker during implantation or subsequent lead location and/or orientation determination and analysis during post-operative programming. This first fringe sensing electrode 126 may include multiple segments of characteristics that are visually distinguishable from each other, such as having unique or complementary shapes, orientations, and/or positions, and these segments may be constructed of a radiopaque biocompatible conductive material to serve as radiopaque orientation markers, as further discussed below. It will be appreciated that because the fringe sensing electrodes are only providing sensing and not stimulation, various visually distinguishable characteristics such as shapes may be chosen for orientation purposes without the limiting bounds for edge effects causing stimulation charge build-up. While a conventional radiopaque marker is enclosed by the lead body 105, the fringe sensing electrodes including the first fringe sensing electrode 126 are on the surface of the lead body 105 and engage surrounding tissue of the patient. Thus, the fringe sensing electrodes may be attached in the same manner that segmented electrodes are typically attached to the lead body 105.

A second fringe sensing electrode 128 that is also ultimately directly coupled to the sensing module 124 resides in a most distal location on the distal end of the lead 104. The second fringe sensing electrode 128 may serve as a distal tip of the lead 104 by being mounted to the very distal end of the lead body 105. Additionally, the second fringe sensing electrode 128 may be constructed of a radiopaque biocompatible conductive material like that of the first fringe sensing electrode to serve as a radiopaque marker during implantation or subsequent lead location and/or orientation determination and analysis. This second fringe sensing electrode 128 may also be constructed of a radiopaque biocompatible conductive material to serve as a radiopaque orientation marker, as further discussed below.

Distal electrodes such as a ring (or omnidirectional) electrode 130 and one or more segmented electrodes 132, 134 may be present between the fringe sensing electrodes. These electrodes 130, 132, 134 may be ultimately directly connected to the switching module 120, so that the electrodes 130, 132, 134 including the individual segments of the segmented electrodes 132, 134, may be assigned as either stimulation electrodes and/or sensing electrodes. It will be appreciated that while the segmented electrodes 132, 134 are separated longitudinally while being immediately adjacent, other combinations are possible such as where a ring or omnidirectional electrode replaces one of the segmented electrodes 132, 134 or is positioned between the segmented electrodes 132, 134,

The distal electrodes of the lead 104 are coupled to corresponding conductors 118 that extend the length of the lead to then couple to proximal connectors present within a header 108 of the implantable medical device 102 where the proximal end of the lead 104, or lead extension when present, is inserted. Conductors of the electrodes 130, 132, 134 form a first plurality of conductors that couple to a first plurality of proximal connectors 114 on the proximal end of the lead 104. The proximal connectors 114 are signal coupled, electrically or otherwise, to a first plurality of contacts 110 of the implantable medical device 102, where this first plurality of electrical contacts 110 are signal coupled, electrically or otherwise, directly to the switching module 120. The first and second plurality of both device contacts 110, 112 and lead connectors 114, 116 may be arranged and physically spaced in distinct groupings or furcates as shown or may be arranged into a single cohesive collection (e.g., such as a connector block).

Conductors of the fringe sensing electrodes 126, 128 form a second plurality of conductors that couple to a second plurality of proximal connectors 116 on the proximal end of the lead 104. The proximal connectors 116 are signal coupled, electrically or otherwise, to a second plurality of electrical contacts 112 of the implantable medical device 102, where this second plurality of electrical contacts 112 are signal coupled, electrically or otherwise, directly to the sensing module 124.

It will be appreciated that the electrical pathways from the electrical contacts 110, 112 to the switching module 120 and the sensing module 124, respectively, pass from the header 108 to inside a housing 109 of the implantable medical device 102. The housing 109 hermetically encloses the circuitry including the power supply, controllers, memory, telemetry module, the switching module 120, the stimulation module 122, and the sensing module 124 to prevent the ingress of any bodily fluids. The electrical pathways from the contacts 110, 112 pass through a feedthrough that provides a hermetic seal between the header 108 and the housing 109. The feedthrough may be a filtered feedthrough to remove spurious energy such as unwanted radio frequency energy from ambient conditions such as a magnetic resonance imaging (MM) scan to contribute to Mill conditional safety of the implantable medical system 100.

In some examples the switching module 120 could be configured to signal connect, electrically or otherwise, a contact of the implantable medical device 102 that corresponds to a desired segment of the segmented electrode to the stimulation module and after delivery of the programmed stimulus, switch the same contact to the sensing module for sensing/recording (e.g., a biomarker signal, resonant response, evoked potential etc.) after a stimulus artifact blanking period.

While the example of FIG. 1 shows certain modules of the implantable medical device 102, it will be appreciated that additional modules and components may also be present. For instance, an on-board power source such as a non-rechargeable or rechargeable battery may be present, a communications module for establishing wireless communications may be present, a processor and associated memory may be present, additional sensors for movement, temperature and the like may be present, and so forth.

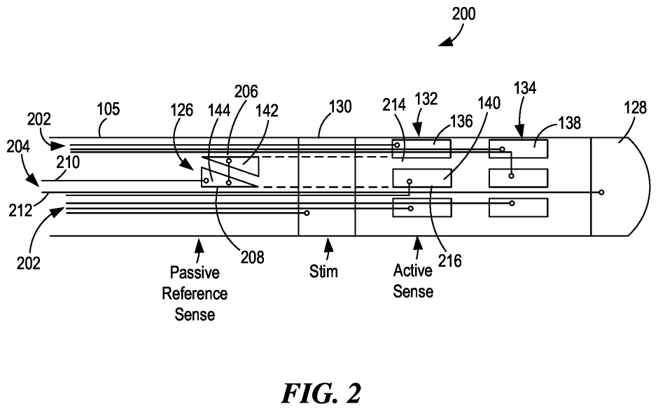

FIG. 2 shows an example of a particular distal end configuration 200 of the lead 104. FIG. 2 further illustrates the first plurality of conductors 202 within the lead body that extend from the first plurality of proximal connectors 114 to the electrodes 130, 132, 134. FIG. 2 also shows the second plurality of conductors 204 within the lead body that extend from the second plurality of proximal connectors 112 to the fringe sensing electrodes 126, 128.

In this particular example, the first fringe sensing electrode 126 includes multiple fragments 142, 144. One or both of these fragments is connected to a first conductor 210 of the second plurality of conductors 204. For instance, these fragments 142, 144 may be connected together such as with a jumper conductor between them and then one of them is further connected to the conductor 210. This allows the first fringe sensing electrode 126 to act as the electrical reference when the sensing module 124 is sensing the biomarker signal according to one sensing montage. Furthermore, as can be seen, the two fragments 142, 144 (which can be placed circumferentially around the longitudinal axis of the lead body and at the same or different longitudinal positions along the longitudinal axis of the lead body) have different visually distinguishable characteristics from one another in that one is the complement of the other. This allows these two fragments 142, 144 to act as lead orientation markers for purposes of identifying the respective positions of the individual segments of the segmented electrodes 132, 134.

To provide orientation guidance, the two fragments 142, 144 each have an orientation feature, in this example a longitudinal edge 206, 208 that aligns circumferentially with a corresponding aspect, such as edge 214, 216, of electrode segments 136, 140, respectively, of the segmented electrode 132. Note that the segment of the segmented electrodes 132, 134 are separated circumferentially around the lead body 105, with at least one edge located at different circumferential positions. Dashed lines are shown for purposes of demonstrating this alignment of the feature of the fragments 142, 144 to the aspects of the electrode segments 136, 140. Therefore, looking at the lead 104 laterally as shown in FIG. 2 via radiological imaging such as an X-ray or fluoroscopy, one can see that the orientation marker with circumferential side on the distal, i.e., fragment 142, has its longitudinal side high. This indicates that the electrode segment 136, which is known to have its edge 214 align with the longitudinal edge 206 of fringe sensing electrode fragment 142, is at the top versus being one of the other electrode segments located elsewhere. Likewise, one can see that the orientation marker with circumferential side on the proximal, i.e., fragment 144, has its longitudinal side low. This indicates that the electrode segment 140, which is known to have its edge 216 align with the longitudinal edge 208 of fringe sensing electrode fragment 144, is at the middle position on the side of the lead 104 being viewed versus being one of the other electrode segments located elsewhere, such as at the top or bottom or backside. Thus, this information may be used to assign one of primary orientation directions (anterior, posterior, medial, or lateral) or second orientation directions (anterio-medial, anterio-lateral, posterior-medial, or posterior-lateral) with respect to patient specific anatomy.

The distal end configuration 200 of FIG. 2 also includes the fringe sensing electrode 128 at the distal tip. In this example, the fringe sensing electrode 128 does not act as an additional radiopaque orientation marker but still acts as a radiopaque depth marker to show where the distal tip is located. This is useful in deep brain stimulation contexts to see where the end of the lead 104 is located relative to adjacent areas of the brain such as the optical pathway where stimulation should be avoided to prevent visual artifacts (e.g., phosphenes evoked by unintentionally stimulating the optic nerve).

In the stim-sense montage of the example of FIG. 2, the ring electrode 130 may be configured via the switching module 120 to provide stimulation, such as in a unipolar (or monopolar) configuration. The fringe sensing electrode 126 acts as the passive reference electrode in the differential bipolar sensing montage via the direct connection to the sensing module 124. At least one of the segments of the segmented electrode 132 may then be configured via the switching module 120 to act as an active sensing electrode.

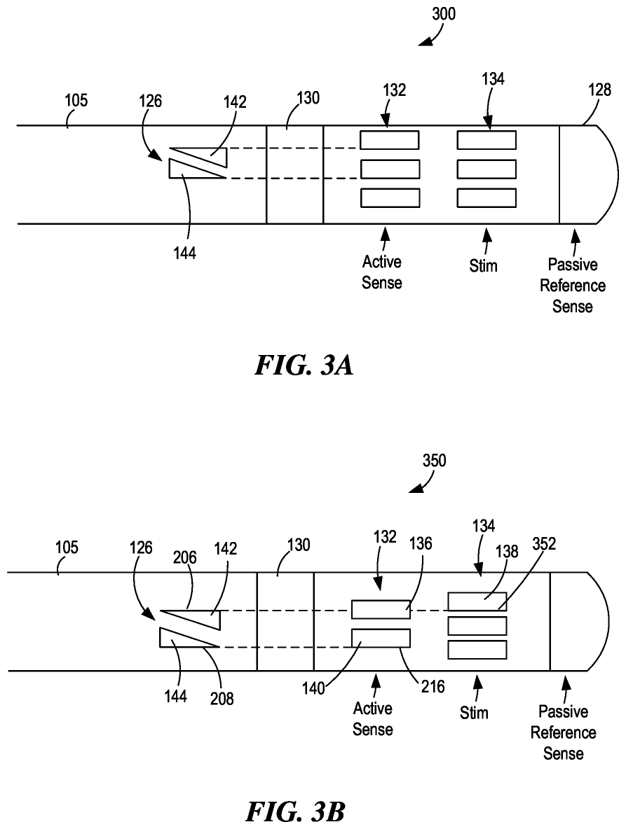

FIG. 3A shows another example of a distal end configuration 300 of the lead 104. Although not shown, the first and second plurality of conductors are present in the same manner shown in FIG. 2. In this example, the fragments 142, 144 of the fringe sensing electrode 126 continue to serve as radiopaque orientation markers for the electrode segments of the segmented electrodes 132, 134. However, the fringe sensing electrode 126 of this configuration 300 is not used as the reference electrode for the sensing montage. Instead, the fringe sensing electrode 128 is configured as the fringe sensing electrode providing a passive reference for the bipolar sensing montage. One of the electrode segments of the segmented electrode 134 is configured by the switching module 120 to be a stimulation electrode in a unipolar (or monopolar) configuration. Therefore, at least one of the electrode segments of the adjacent segmented electrode 132 may be configured by the switching module 120 to be an active sense electrode.

It will be appreciated that many variations are applicable for the distal end configurations. For instance, rather than multiple segmented electrodes 132, 134 (in low-resolution or high-resolution segmented design), a single segmented electrode may be used. In that case, if the single segmented electrode is configured for stimulation, then the distal fringe sensing electrode 128 may be paired with the ring electrode 130 to provide the bipolar sensing montage immediately surrounding the stimulation electrode. As another example, a distal end configuration may include only ring electrodes and no segmented electrodes, such that orientation of segments is not a concern. In that situation, the fringe sensing electrodes 126, 128 may continue to act as passive reference electrodes for sensing by way of direct coupling to the sensing circuit 124 but provide no orientation guidance, although still provide longitudinal position guidance when viewed by X-ray or fluoroscopy.

FIG. 3B shows another example of a distal end configuration 350 of the lead 104. Although not shown, the first and second plurality of conductors are present in the same manner shown in FIG. 2. In this example, the fragments 142, 144 of the fringe sensing electrode 126 continue to serve as radiopaque orientation markers for the electrode segments of the segmented electrodes 132, 134. However, as a departure from the example of FIG. 3A, the segments of the segmented electrode 132 are at different transverse levels than the segments of the segmented electrode 134. Consequently, the edge 206 of fragment 142 aligns with the edge 352 of segment 138 of segmented electrode 134 rather than aligning with an edge of any segment of segmented electrode 132. Edge 208 of fragment 144 aligns with edge 216 of the segment 140 of the segmented electrode 132.

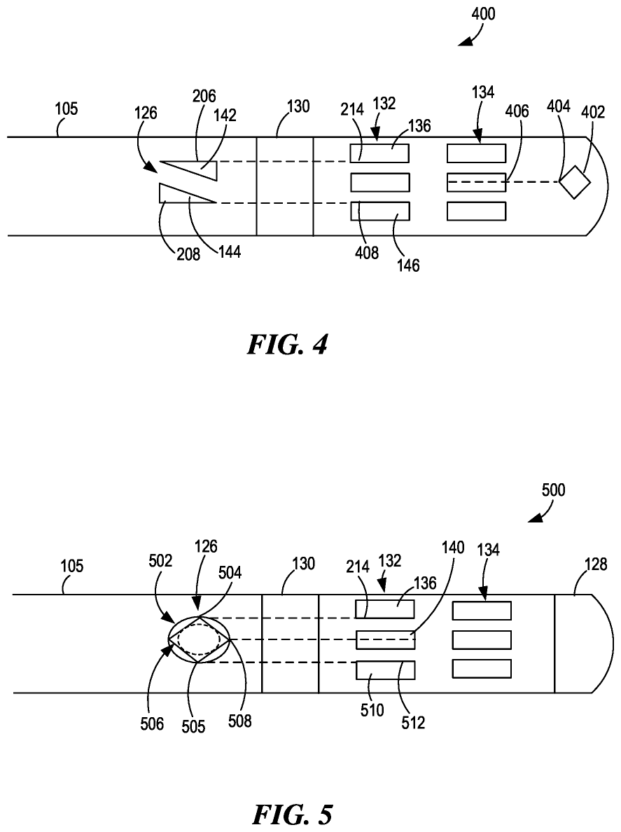

FIG. 4 shows another example of a distal end configuration 400 of the lead 104. Although not shown, the first and second plurality of conductors are present in the same manner shown in FIG. 2. In this example, rather than using a most distal fringe sensing electrode as the distal tip of the lead 104, a most distal fringe sensing electrode 402 is included as an additional radiopaque orientation marker. The fringe sensing electrode 402 includes a shape with a feature 404, such as the most proximal and/or distal point(s) or features of a diamond shape, that is/are in alignment with an aspect such as the center of a particular electrode segment 406 of the segmented electrode 134. This provides an additional visual point or feature of reference for determining the orientation of the electrode segments of the segmented electrode 134 when viewing the implanted lead 104 laterally with an X-ray or fluoroscopy. Likewise, circumferentially spaced points or features of the diamond shape of feature 404 may provide a potential reference aid as well when determining the relative positioning of the lead and electrodes thereon. As with the prior examples, the fringe sensing electrode 402 may be directly coupled to the sensing circuit 124 and serve as a passive reference electrode in a bipolar sensing montage.

FIG. 5 shows another example of a distal end configuration 500 of the lead 104. In this example, the proximal fringe sensing electrode 126 is configured differently. Here, the fringe sensing electrode 126 includes fragments 502 and 506 where fragment 502 is a circle with a central hole and fragment 506 is a diamond shape. The fragments 502, 506 are present circumferentially opposite of one another. The fragment 502 includes a feature that is a point 504 whose tangent aligns with the edge 214 of electrode segment 136 of the segmented electrode 132. The fragment 502 also includes a feature that is a point 505 whose tangent aligns with the edge 512 of electrode segment 510 of the segmented electrode 132. The fragment 502 includes a feature that is a point 508 that aligns with the center of electrode segment 140 of the segmented electrode 132. As shown in FIG. 5, the two fragments 502 and 506 are overlapping in the lateral view due to being on opposite sides, but the relative position of the circle shape to the diamond shape allows a quick confirmation via X-ray or fluoroscopy of segmented electrode orientation throughout the potential 360 degrees of lead circumferential orientation variations. As is well known, the structure that the x-ray beam strikes first because of being closest to the x-ray source will be magnified in relation to those nearer the detector, thus allowing orientation to be determined from the overlapping fragments 502 and 506.

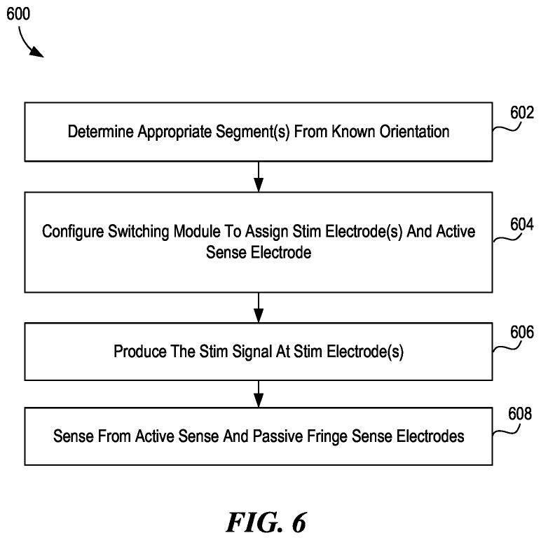

An example of a sequence of operations 600 that may be performed for the implantable medical system 100 that includes the fringe sensing electrodes is shown in FIG. 6. Where the electrodes between the fringe sensing electrodes include segmented electrodes and where radiopaque orientation markers are present, such as where at least one of the fringe sensing electrodes serves as the radiopaque orientation marker, the segments to use for stimulation and active sensing are determined based on the orientation at an operation 602. Here, a clinician may make this determination when programming the implantable medical device 100 based on the known segmented electrode orientation and location with respect to the patient specific atlas or relevant regional anatomy. In some instances, the orientation could be determined post-operatively after the electrode-tissue interface has been formed for e.g., a few days or weeks after the lead implantation procedure.

The implantable medical device 102, via an on-board controller that may be integral to the switching module 120 or may be a stand-alone controller, then assigns the desired electrode for stimulation and the desired electrode for active sensing by configuring the switching module 120 at an operation 604. The configured switching module 120 connects the desired segmented electrode for stimulation to the stimulation module 122 and connects the desired active sensing electrode to the sensing module 124. To achieve this configuration, instructions may be received at the implantable medical device via a telemetry signal from an external programmer being programmed by the clinician who has observed the orientation of the segmented electrodes, if any. In some instances, the device can be configured to capture events enabled by a patient controller (or programmer). In case an event capture trigger has been received, the device then connects a segmented or ring electrode currently configured to deliver stimulation for sensing by reconfiguring the switching module. In addition to orientation, determination of the appropriate segment(s) for stimulation may be on the basis of a sensed biomarker signal of interest ascertaining source localization or orientation to be localized on to a particular segment of the segmented electrode.

The external programmer may also provide telemetry signals to further program aspects of the stimulation signal parameters such as amplitude, pulse width, pulse interval, and the like. The stimulation module 122 of the implantable medical device 102 then produces the stimulation waveform that are delivered from the programmed stimulation electrode(s) configuration to the tissue of the patient at an operation 606. Because the fringe sensing electrodes are present on the lead and out flank all of the potential stimulation electrodes, any stimulation electrode is available for designation as the sense and stimulation electrode to provide the stimulation signal while still being able to sense simultaneously.

The sensing module 124 then senses biomarker signals via the active sensing electrode and the fringe sensing electrode acting as the passive reference for differential bipolar montage sensing at an operation 608. As discussed above, the sensing electrodes surround the stimulation electrode(s) as it is likely that the resulting biomarker will be produced in close proximity to the stimulation electrode that provided the stimulation signal to the tissue of the patient.

It will be appreciated that prior to the operations shown for FIG. 6, sensing may be performed to obtain a baseline biomarker. This baseline biomarker may then be compared with the biomarker response obtained pursuant to the sensing of operation 608 to ascertain if a specific characteristic of the sensed signal can be influenced with applied stimulation. For example, a specific characteristic of the sensed signal relative to the baseline could be a change in power of the signal in a specific frequency band such as reduction in the power of the beta band.

While embodiments have been particularly shown and described, it will be understood by those skilled in the art that various other changes in the form and details may be made therein without departing from the spirit and scope of the invention.

What is claimed is:

1. An implantable medical lead configured for implantation within a patient, comprising:
a lead body;
a first plurality of proximal contacts on a proximal end of the lead body;
a second plurality of proximal contacts on the proximal end of the lead body;
at least one distal electrode on a distal end of the lead body, the at least one distal electrode comprising a segmented electrode having segments separated circumferentially about the lead body with each of the segments having at least one edge at circumferential positions about the lead body;
a first sensing electrode on the distal end of the lead body, the sensing electrode being radiopaque and being located at a first longitudinal position of the distal end of the lead body relative to the at least one distal electrode, the first sensing electrode comprising electrode fragments having characteristics that are visually distinguishable from each other and where the characteristics have an orientation feature that aligns circumferentially with an aspect of a corresponding one of the segments of the segmented electrode;
a second sensing electrode located at a second longitudinal position of the distal end of the lead body so that the at least one distal electrode is longitudinally between the first sensing electrode and the second sensing electrode;
a first plurality of conductors surrounded by the lead body and extending longitudinally, each conductor of the first plurality of conductors interconnecting one of the proximal contacts of the first plurality of proximal contacts to one of the segments of the segmented electrode on the distal end of the lead body; and
a second plurality of conductors surrounded by the lead body and extending longitudinally, a first conductor of the second plurality of conductors interconnecting one of the proximal connectors of the second plurality of proximal connectors to the first sensing electrode and a second conductor of the second plurality of conductors interconnecting another one of the proximal connectors of the second plurality of proximal connectors to the second sensing electrode.

2. The implantable medical lead of claim 1, wherein the first sensing electrode is proximal of the second sensing electrode.

3. The implantable medical lead of claim 1, wherein the second sensing electrode has a shape that has an orientation feature that aligns circumferentially with an aspect of a corresponding one of the segments of the segmented electrode.

4. The implantable medical lead of claim 1, wherein the electrode fragments of the first sensing electrode are electrically connected together.

5. The implantable medical lead of claim 1, wherein the first and second sensing electrodes comprise platinum-iridium.

6. An implantable medical system configured to provide stimulation and sensing within a patient, comprising:
an implantable medical device comprising a stimulation module, a sensing module, a first plurality of contacts signal coupled to the stimulation module, and a second plurality of contacts coupled to the sensing module; and
an implantable medical lead comprising:
a lead body;
a first plurality of proximal connectors on a proximal end of the lead body, each proximal connector of the first plurality of proximal connectors being coupled to a corresponding proximal contact that is coupled to the stimulation module; a second plurality of proximal connectors on the proximal end of the lead body, each proximal connector of the second plurality of proximal connectors being coupled to a corresponding proximal contact that is coupled to the sensing module;
at least one distal electrode on a distal end of the lead body, the at least one distal electrode comprising a segmented electrode having segments separated circumferentially about the lead body with each of the segments having at least one edge at circumferential positions about the lead body;
a first sensing electrode on the distal end of the lead body, the first sensing electrode being radiopaque and being located at a first longitudinal position of the distal end of the lead body relative to the at least one distal electrode, the first sensing electrode comprising electrode fragments having characteristics that are visually distinguishable from each other and where the characteristics have an orientation feature that aligns circumferentially with an aspect of a corresponding one of the segments of the segmented electrode;
a second sensing electrode located at a second longitudinal position of the distal end of the lead body so that the at least one distal electrode is longitudinally between the first sensing electrode and the second sensing electrode;
a first plurality of conductors surrounded by the lead body and extending longitudinally, each conductor of the first plurality of conductors interconnecting one of the proximal contacts of the first plurality of proximal contacts to one of the segments of the segmented electrode on the distal end of the lead body; and
a second plurality of conductors surrounded by the lead body and extending longitudinally, a first conductor of the second plurality of conductors interconnecting one of the proximal connectors of the second plurality of proximal connectors to the first sensing electrode and a second conductor of the second plurality of conductors interconnecting another one of the proximal connectors of the second plurality of proximal connectors to the second sensing electrode.

7. The implantable medical system of claim 6, further comprising a switching module coupled to the stimulation module and electrically coupled to the first plurality of contacts, and wherein the switching module provides the electrical coupling of the first plurality of contacts to the stimulation module.

8. The implantable medical system of claim 6, wherein the first sensing electrode is proximal of the second sensing electrode.

9. The implantable medical system of claim 6, wherein the second sensing electrode has a shape that has an orientation feature that aligns circumferentially with an aspect of a corresponding one of the segments of the segmented electrode.

10. The implantable medical system of claim 6, wherein the electrode fragments of the first sensing electrode are electrically connected together.

11. The implantable medical system of claim 6, wherein the first and second sensing electrodes comprise platinum-iridium.

12. An implantable medical system configured to provide stimulation and sensing within a patient, comprising:
an implantable medical device comprising a stimulation module, a sensing module, a switching module coupled to the stimulation module and the sensing module, a plurality of contacts signal coupled to the switching module, and a plurality of contacts coupled to the sensing module; and
an implantable medical lead comprising:
a lead body;
a first plurality of proximal connectors on a proximal end of the lead body, each proximal connector of the first plurality of proximal connectors being coupled to a corresponding proximal contact that is coupled to the switching module;
a second plurality of proximal connectors on the proximal end of the lead body, each proximal connector of the second plurality of proximal connectors being coupled to a corresponding proximal contact that is directly coupled to the sensing module;
at least one distal electrode on a distal end of the lead body, the at least one distal electrode comprising a segmented electrode having segments separated circumferentially about the lead body with each of the segments having at least one edge at circumferential positions about the lead body;
a first sensing electrode on the distal end of the lead body, the sensing electrode being located at a first longitudinal position of the distal end of the lead body relative to the at least one distal electrode;
a second sensing electrode located at a second longitudinal position of the distal end of the lead body so that the at least one distal electrode is longitudinally between the first sensing electrode and the second sensing electrode; and
a first plurality of conductors surrounded by the lead body and extending longitudinally, each conductor of the first plurality of conductors interconnecting one of the proximal contacts of the first plurality of proximal contacts to one of the segments of the segmented electrode on the distal end of the lead body; and
a second plurality of conductors surrounded by the lead body and extending longitudinally, a first conductor of the second plurality of conductors interconnecting one of the proximal connectors of the second plurality of proximal connectors to the first sensing electrode and a second conductor of the second plurality of conductors interconnecting another one of the proximal connectors of the second plurality of proximal connectors to the second sensing electrode.

13. The implantable medical system of claim 12, wherein the first sensing electrode comprises electrode fragments having characteristics that are visually distinguishable from each other and where the characteristics have an orientation feature that aligns circumferentially with an aspect of a corresponding one of the segments of the segmented electrode.

14. The implantable medical system of claim 13, wherein the second sensing electrode has a shape that has an orientation feature that aligns circumferentially with an aspect of a corresponding one of the segments of the segmented electrode.

15. The implantable medical system of claim 12, wherein the first sensing electrode is proximal of the second sensing electrode.

16. The implantable medical system of claim 12, wherein the electrode fragments of the first sensing electrode are electrically connected together.

17. The implantable medical system of claim 12, wherein the first and second sensing electrodes comprise platinum-iridium.

18. A method of providing stimulation and sensing within a patient, comprising:
providing an implantable medical device comprising a stimulation module, a sensing module, a plurality of contacts signal coupled to the stimulation module, and a plurality of contacts coupled to the sensing module;
providing an implantable medical lead comprising:
a lead body;
a first plurality of proximal connectors on a proximal end of the lead body, each proximal connector of the first plurality of proximal connectors being coupled to a corresponding proximal contact that is coupled to the stimulation module;
a second plurality of proximal connectors on the proximal end of the lead body, each proximal connector of the second plurality of proximal connectors being coupled to a corresponding proximal contact that is coupled to the sensing module;
at least one distal electrode on a distal end of the lead body, the at least one distal electrode comprising a segmented electrode having segments separated circumferentially about the lead body with each of the segments having at least one edge at circumferential positions about the lead body;
a first sensing electrode on the distal end of the lead body, the first sensing electrode being radiopaque and being located at a first longitudinal position of the distal end of the lead body relative to the at least one distal electrode, the first sensing electrode comprising electrode fragments having characteristics that are visually distinguishable from each other and where the characteristics have an orientation feature that aligns circumferentially with an aspect of a corresponding one of the segments of the segmented electrode;
a second sensing electrode located at a second longitudinal position of the distal end of the lead body so that the at least one distal electrode is longitudinally between the first sensing electrode and the second sensing electrode; and
a first plurality of conductors surrounded by the lead body and extending longitudinally, each conductor of the first plurality of conductors interconnecting one of the proximal contacts of the first plurality of proximal contacts to one of the segments of the segmented electrode on the distal end of the lead body; and
a second plurality of conductors surrounded by the lead body and extending longitudinally, a first conductor of the second plurality of conductors interconnecting one of the proximal connectors of the second plurality of proximal connectors to the first sensing electrode and a second conductor of the second plurality of conductors interconnecting another one of the proximal connectors of the second plurality of proximal connectors to the second sensing electrode;
determining which segment of the segmented electrode to provide a stimulation signal based on the location of the orientation feature of each of the electrode fragments of the first sensing electrode;
providing the stimulation signal from the stimulation module to the determined segment of the segmented electrode; and
sensing at the sensing module for a biomarker signal occurring between the first sensing electrode and the second sensing electrode.

19. The method of claim 18, further comprising determining an orientation of the segmented electrode within the brain based on position of the orientation feature as revealed by radiological imaging.

20. The method of claim 18, wherein providing the implantable medical device further comprises providing a switching module coupled to the stimulation module and electrically coupled to the first plurality of contacts, and wherein the switching module provides the electrical coupling of the first plurality of contacts to the stimulation module.

21. The method of claim 18, wherein the first sensing electrode is proximal of the second sensing electrode.

22. The method of claim 18, wherein the second sensing electrode has a shape that has an orientation feature that aligns circumferentially with an aspect of a corresponding one of the segments of the segmented electrode.

23. The method of claim 18, wherein the electrode fragments of the first sensing electrode are electrically connected together.

24. The method of claim 18, wherein the first and second sensing electrodes comprise platinum-iridium.

25. A method of providing stimulation and sensing within a patient, comprising:
providing an implantable medical device comprising a stimulation module, a sensing module, a switching module coupled to the stimulation module and the sensing module, a plurality of contacts signal coupled to the switching module, and a plurality of contacts coupled to the sensing module;
providing an implantable medical lead comprising:
a lead body;
a first plurality of proximal connectors on a proximal end of the lead body, each proximal connector of the first plurality of proximal connectors being coupled to a corresponding proximal contact that is coupled to the switching module;
a second plurality of proximal connectors on the proximal end of the lead body, each proximal connector of the second plurality of proximal connectors being coupled to a corresponding proximal contact that is directly coupled to the sensing module;
at least one distal electrode on a distal end of the lead body, the at least one distal electrode comprising a segmented electrode having segments separated circumferentially about the lead body with each of the segments having at least one edge at circumferential positions about the lead body;
a first sensing electrode on the distal end of the lead body, the first sensing electrode being located at a first longitudinal position of the distal end of the lead body relative to the at least one distal electrode;
a second sensing electrode located at a second longitudinal position of the distal end of the lead body so that the at least one distal electrode is longitudinally between the first sensing electrode and the second sensing electrode; and
a first plurality of conductors surrounded by the lead body and extending longitudinally, each conductor of the first plurality of conductors interconnecting one of the proximal contacts of the first plurality of proximal contacts to one of the segments of the segmented electrode on the distal end of the lead body; and
a second plurality of conductors surrounded by the lead body and extending longitudinally, a first conductor of the second plurality of conductors interconnecting one of the proximal connectors of the second plurality of proximal connectors to the first sensing electrode and a second conductor of the second plurality of conductors interconnecting another one of the proximal connectors of the second plurality of proximal connectors to the second sensing electrode;
configuring the switching module to signal connect a contact of the implantable medical device that corresponds to a desired segment of the segmented electrode to the stimulation module;
providing a stimulation signal from the stimulation module to the desired segment of the segmented electrode; and
sensing at the sensing module for a biomarker signal occurring between the first sensing electrode and the second sensing electrode.

26. The method of claim 25, wherein the first sensing electrode comprises electrode fragments having characteristics that are visually distinguishable from each other and where the characteristics have an orientation feature that aligns circumferentially with an aspect of a corresponding one of the segments of the segmented electrode.

27. The method of claim 26, further comprising:
determining an orientation of the segmented electrode within the brain based on position of the orientation feature as revealed by radiological imaging; and
determining which segment of the segmented electrode is desired based on the determined orientation.

28. The method of claim 25, wherein the second sensing electrode has a shape that has an orientation feature that aligns circumferentially with an aspect of a corresponding one of the segments of the segmented electrode.

29. The method of claim 25, wherein the first sensing electrode is proximal of the second sensing electrode.

30. The method of claim 25, wherein the electrode fragments of the first sensing electrode are electrically connected together.

31. The method of claim 25, wherein the first and second sensing electrodes comprise platinum-iridium.
